(19) United States
(12) Patent Application Publication
(10) Pub. No.: US 2003/0050605 A1
(43) Pub. Date: Mar. 13, 2003

(54) Closure assembly in particular for hypodermic syringes

(76) Inventor: John Targell, Craigie Byre Craigie Kilmarnock Ayrshire (GB)

Correspondence Address: BAKER & DANIELS, 111 E. WAYNE STREET, SUITE 800, FORT WAYNE, IN, 46802

(21) Appl. No.: 10/149,342

(22) PCT Filed: Dec. 1, 2000
(86) PCT No.: PCT/GB00/04573

(51) Int. Cl. A61M 5/32

(52) U.S. Cl. 604/192; 604/200

(57) ABSTRACT

A closure member comprises a rim portion defining an aperture and a blocking portion which closes the aperture but can be broken free from the run portion, the portions having a zone of intimate contact therebetween produced in a moulding process (e.g. a two shot moulding process) to provide a hermetic seal between the portions. The two portions are also be coupled together at the zone of contact in such a way that the blocking portion is only removable upon application of a predetermined load thereto.

This invention relates to a closure assembly for use in providing a seal, especially a hermetic seal, the closure assembly comprising a portion which can be broken away to provide an aperture.

An important, though by no means exclusive, application for such closure assemblies is in a hypodermic syringe of the kind which provides for retraction of the needle after use into the body of the plunger. To this end, the forward end wall of the plunger can be provided with a so-called “bursting disc” which breaks away at the end of the injection stroke to provide an aperture through which the needle may pass into the body of the plunger. One form of such syringe is described in our prior EP-A-776225.

Typically the plunger is moulded from a plastics material and the “bursting disc” is defined by a circular line of weakness in the forward end wall of the plunger. To ensure fracture at the line of weakness, it is necessary to mould the “bursting disc” component from a brittle material such as polystyrene which imposes restrictions on the range of materials that can be used.

According to one aspect of the present invention there is provided a closure assembly comprising a rim portion defining an aperture and a removable blocking portion which closes the aperture, said portions being united in sealing contact with each other at a moulding interface which extends around the blocking portion.

The arrangement is preferably such that the two portions are united together by the moulding process to afford a well-defined threshold at which the blocking portion breaks away from the rim portion in response to the application of mechanical force to the blocking portion.

Preferably the arrangement is such that the blocking portion breaks away cleanly from the rim portion at said interface to leave a dimensionally well-defined aperture.

Preferably one of the portions is produced as a plastics moulding in a mould having moulding surfaces defined, in part, by the other portion so as to provide said moulding interface.

Conveniently both portions are produced as plastics mouldings, one portion being produced in advance of the other so that moulding of the other portion can be effected with said one portion in situ.

The two portions may be of plastics materials having different chemical compositions and/or characteristics or they may both be of the same plastics material.

The blocking portion may be capable of being broken away from the rim portion without deformation of either of the two portions and, to this end, said interface may be shaped so that no deformation of either part occurs when breaking away the blocking portion by loading the same relative to the rim portion in at least one direction.

Alternatively, the two portions may be interlocked with each other so that some deformation of at least one of the parts occurs when breaking away the blocking portion.

Preferably at the moulding interface, an annular surface of one of said portions is conformed with an annular surface of the other portion in the course of moulding said one portion.

The annular surfaces may be cylindrical or of other configuration, e.g. conical.

At the moulding interface, the portions may intimately contact each other with a degree of fusion bonding consistent with securing fracture preferentially at the interface region when the blocking portion is caused to break away from the rim portion.

The assembly preferably includes means for resisting removal of the blocking portion so that the blocking portion can only be separated from the rim portion by application of axial load of predetermined magnitude.

Such means may comprise a detent arrangement acting between the portions. The detent arrangement may be provided at or in the vicinity of said moulding interface. For example, the rim and blocking portions may, by virtue of the mould design, have interengaging elements which lock the portions together to prevent separation of the portions, at least one of the elements being resiliently deformable to allow the disengagement of the elements on application of sufficient loading to the blocking portion relative to the rim portion.

Resistance to separation of the portions may additionally or alternatively be afforded by the nature of the interaction between the rim and blocking portions at the moulding interface. For instance, there may a shrink type fit between the portions at the moulding interface obtained by material shrinkage during cooling following the moulding process.

As a further addition or alternative to the detent arrangement and/or shrink type fit mentioned above, resistance to separation may be provided by fusion bonding between said portions at the moulding interface. Such fusion bonding may range from relatively weak, e.g. as a result of some degree of diffusion of material from one portion across the moulding interface into the other portion, through to relatively strong, including for example significant welding together of the portions at the moulding interface.

Depending on the nature of interaction desired between the rim and blocking portions at the moulding interface, the material or materials from which said portions are produced may be selected so as to secure the desired extent of fusion bonding, if any. For example, if negligible fusion bonding is desired, the materials employed will usually differ in chemical and/or physical characteristics and will be such that no significant diffusion of material takes place across the moulding interface as a result of the moulding process. Similarly the materials can be appropriately selected to obtain the desired degree of fusion bonding where there is a requirement to develop a fusion bond at the moulding interface.

In the latter case for example, the rim and blocking portions may be composed of the same material so that significant fusion of the material takes place between the two portions.

The width of the moulding interface and hence the zone of contact between the two portions may be selected with regard to the required effectiveness of the seal and/or with regard to the resistance to separation of the blocking portion required at said interface. For instance, a given degree of resistance may be secured by a relatively narrow interface where the portions are strongly fusion bonded together whereas a wider interface may be required if resistance is afforded by shrinkage and/or a weak fusion bond.

Preferably the closure assembly is produced by a two-shot moulding process in which one portion, e.g. the blocking portion, is initially formed and located so that an annular surface thereof forms a boundary surface of the mould cavity in which the other portion is produced so that, during formation of the second portion, an annular surface of the latter is conformed with the annular surface of the first portion.

Because the closure assembly of the present invention is not limited to fabrication using brittle materials such as styrene, a wider range of options are available for material selection.

The closure assembly may be a “single-use” arrangement—i.e. the blocking portion is irreversibly removable from the rim portion so that, once the two portions have been separated from one another and the seal broken, they cannot be restored to the same condition of sealing and separation resistance simply by re-inserting the blocking portion into the rim portion.

The closure assembly may be used in a variety of applications requiring a fluid seal which can be readily broken upon application of force—for instance, the closure assembly according to the invention may be employed in the sealing of drinks containers. Also the closure assembly can be used as a so-called bursting disc which ruptures by separation of the blocking portion from the rim portion in response to the application of load or pressure. It will be appreciated that the blocking portion need not necessarily be disc-shaped when used in a bursting disc-type device.

The closure member is particularly useful in a fluid handling device, e.g. a syringe, as described in EP-A-776225.

Thus, according to a second aspect of the present invention there is provided a fluid handling device having a hollow needle for dispensing fluid and a needle retraction assembly for transferring the needle after use into a housing of the device, the housing being provided with a closure assembly as defined hereinabove, the aperture defined by the rim portion constituting a needle entry aperture which, prior to operation of the needle retraction assembly, is closed by the blocking portion, the blocking portion being broken away during needle retraction to allow passage of the needle into the housing via said opening.

The sealing effected at said interface serves to prevent ingress or egress of fluid into the housing from a fluid holding chamber of the device.

The closure assembly may be fitted to the housing or alternatively the rim portion of the closure assembly may be integrally connected with the housing.

The housing preferably forms a plunger or piston received within a tubular body portion of the device which acts as a fluid-holding reservoir in use and the blocking portion when held captive within the aperture may project forwardly of the remainder of the housing for co-operation with part of the needle retraction mechanism.

The invention will now be described by way of example only with reference to the accompanying drawings in which:

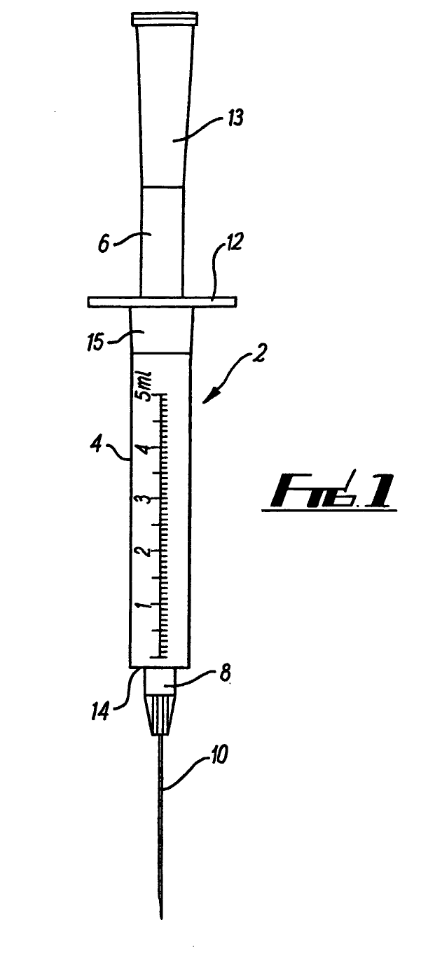

FIG. 1 is a view of a syringe in accordance with the invention in its charged condition prior to use;

FIG. 2 is a fragementary sectional view on an enlarged scale illustrating the relative positioning of a piston and body portion of the syringe part-way towards completion of the dispensing action;

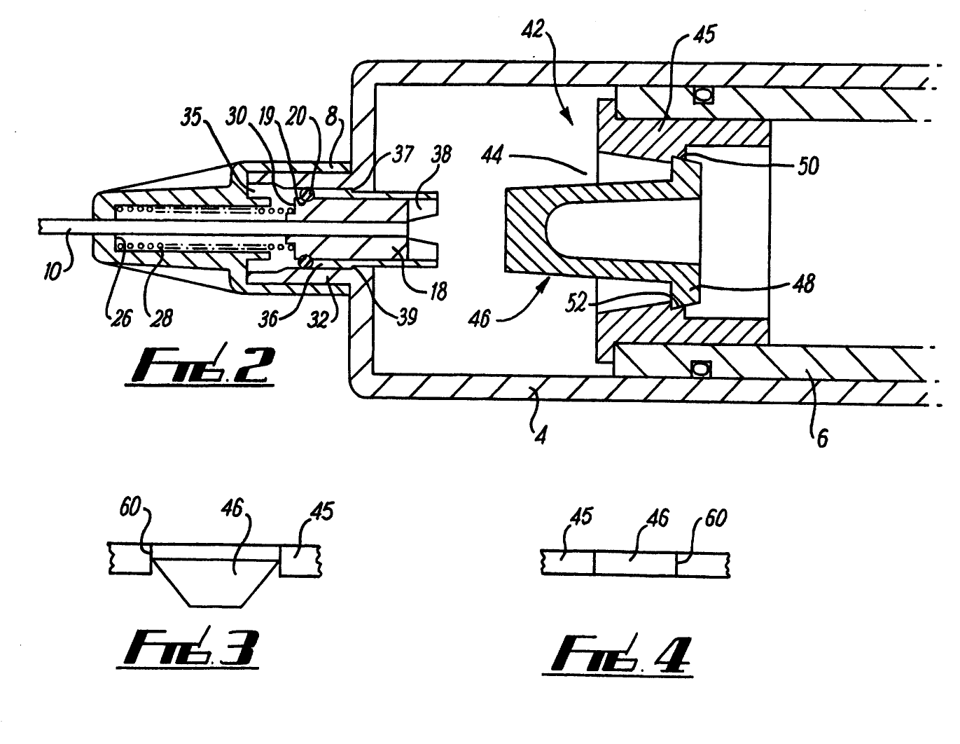

FIG. 3 is a diagrammatic view showing a modified zone of contact between the rim and blocking portions; and

FIG. 4 is a diagrammatic view of a disc-type closure assembly.

The disposable syringe shown in the drawings may be generally similar to that described and illustrated in our prior EP-A-776225. Specifically, apart from the design of the leading end of the piston, the needle retraction mechanism is essentially the same as that described in EP-A-776225 and reference should therefore be made to EP-A-776225 for a detailed description of the syringe and its operation.

Referring now to the drawings, the disposable syringe 2 comprises a hollow body portion 4, from the rear of which (upper end as viewed in FIG. 1) protrudes a piston 6. At the lower end of the body portion 4 is provided a mounting portion 8 for a needle 10, having a through passage at the inner end of which is an upstanding annular wall 9.

The piston 6 is hollow having its trailing end closed by means of a cap (not shown). The forward end of the piston is arranged to receive a closure assembly 42 which engages firmly in the piston to form an end wall of the piston while defining an aperture 44 which is sufficiently large to allow the needle and mounting portion 8 to pass through during needle retraction. The insert 42 comprises a rim portion 45 fitted with a forwardly projecting blocking portion 46 of generally frusto-conical configuration so that the blocking portion 46 closes the aperture 44. The blocking portion 46 and rim portion 45 seal the forward end of the piston to prevent ingress of liquid into the piston interior from the interior of body portion 4.

The blocking portion 46, by virtue of moulding, becomes integrated with, and hence captive with, the ring portion 45. To this end, in the illustrated embodiment the blocking portion is formed with an annular flange 48 having a generally frusto-conical configuration surface 50 which is received within a complementary annular rebate 52 formed in the internal wall of the rim portion 45 thereby forming a detent. The mating conical surfaces of the flange 48 and the rebate 52 converge towards an imaginary apex in the direction of travel of the needle into the interior of the piston during needle retraction as described below. The interengagement between the flange 48 and the rebate 52 renders the blocking portion 46 axially captive to the ring portion 45 with the forward end of blocking portion 46 projecting forwardly of the piston for eventual engagement with the extension 38. Also the mating surfaces of the flange 48 and the rebate 52 form a zone of contact between the rim portion 45 and the blocking portion 45 to provide the above-mentioned seal between the blocking portion 45 and the aperture 44.

In operation, when the piston has been substantially fully depressed, the blocking portion 46 is subjected to loading as a result of contact with the extension 38 and, by appropriate design, when the load reaches a predetermined magnitude, the force on the blocking portion 46 becomes sufficient to dislodge the flange 48 from the rebate 52 so that the blocking portion together with the assembly comprising the needle 10 and the end portion 18 are automatically propelled by spring 28 into the interior of the piston 6, e.g. to avoid a needle stick hazard. Release of the flange 48 from the rebate 52 may be facilitated by resilient deformation of the rim portion and/or blocking portion if these components are produced from resiliently deformable plastics materials such as polyethylene or polypropylene.

The closure assembly comprising parts 45 and 46 is conveniently produced in the course of a two-shot moulding process, for instance a first stage in which the blocking portion is produced followed by a second stage in which the part 46 is moulded onto the blocking portion thereby connecting the two parts together through the interengaging flange and rebate and providing a zone of intimate contact for sealing the aperture 44.

To prevent the parts 42 and 46 welding together inseparably during the moulding process, the two parts may be moulded using materials which are compatible with each other and have different characteristics. However, as previously indicated, some degree of fusion bonding between the rim and closure parts may be desirable, e.g. to ensure sealing and, where desired, to play a role in predetermining the loading necessary to break the blocking portion away from the rim portion.

The closure assembly in FIGS. 1 and 2 is shown fitted into the forward end of the piston 6; however, in an alternative embodiment, the rim portion may be formed integrally with at least the forward end portion of the piston so that the piston and rim portion can be produced as a single moulding. For example, both the piston and rim portion may be made from a plastics material such as polyethylene or polypropylene.

In the embodiment of FIGS. 1 and 2, the blocking portion is held captive by the detent arrangement. However, the detent arrangement may be dispensed with, or it may be supplemented, by resistance developed at the zone of sealing contact, i.e. the moulding interface, between the blocking portion and the rim portion. FIG. 3 illustrates a modification in which blocking portion is rendered captive to the rim portion by virtue of an interference or shrink type fit between the parts at a zone of contact 60 and without the aid of a detent arrangement. As previously mentioned, the shrink fit may be obtained during the moulding process by moulding the rim portion 45 around the blocking portion 46 and exploiting material shrinkage on cooling to secure the interference fit. Where the blocking portion is held captive in this way, there is not necessarily any signficant fusion bonding between the materials although, if desired, the material(s) may be selected so that such fusion bonding is present, e.g. as a result of some degree of diffusion of material between the two parts.

FIG. 4 illustrates diagrammatically a closure assembly with a plate-like or disc-like configuration in which the trailing and forward surfaces of the rim portion 45 and the blocking portion 46 are substantially flush with each other. In the embodiment of FIG. 4, the blocking portion 46 is rendered captive to the rim portion 46 at the zone of contact 60 by the shrinkage and/or fusion bonding mechanisms described above. However, the blocking portion may instead or additionally be held captive by a detent arrangement in the vicinity of the zone of contact 60.

Because the closure assembly of the present invention does not use an integral knock-out section as in EP-A-776225 to create the needle entry aperture, it will be seen that the closure is not limited to brittle materials such as styrene. Because of this increased flexibility, the possibility arises of making the entire safety syringe using materials such as polyethylene and/or polypropylene. The ability to call upon a wider range of fabrication materials may also be advantageous in terms of comparability with the drugs to be dispensed. The manner in which the blocking portion is held captive within the rim portion allows separation of the blocking portion by a predictable and controllable breaking force while securing a clean and dimensionally well-defined break-out aperture thus avoiding interference with needle travel during retraction. In each of the embodiments described above, it will be appreciated that the closure member functions as a bursting disc-type device and whilst, as illustrated in FIG. 4, the blocking portion may be generally disc-shaped it may have other configurations as illustrated in FIGS. 2 and 4 for example.

1. A closure assembly comprising a rim portion defining an aperture and a removable blocking portion which closes the aperture, said portions being united in sealing contact with each other at a moulding interface which extends around the blocking portion.

2. A closure assembly as claimed in claim 1 in which the arrangement is such that the two portions are united together by the moulding process to afford a well-defined threshold at which the blocking portion breaks away from the rim portion in response to the application of mechanical force to the blocking portion.

3. A closure assembly as claimed in claim 1 or 2 in which the arrangement is such that the blocking portion breaks away cleanly from the rim portion at said interface to leave a dimensionally well-defined aperture.

4. A closure assembly as claimed in any one of claims 1 to 3 in which one of the portions is produced as a plastics moulding in a mould having a moulding surface or surfaces defined, in part, by the other portion so as to provide said moulding interface.

5. A closure assembly as claimed in any one of claims 1 to 4 in which both portions are produced as plastics mouldings, one portion being produced in advance of the other so that moulding of the other portion can be effected with said one portion in situ.

6. A closure assembly as claimed in any one of claims 1 to 4 in which both portions are produced as plastics mouldings in a two-shot moulding process.

7. A closure assembly as claimed in any one of claims 1 to 6 in which the two portions are of plastics materials having different chemical compositions and/or characteristics.

8. A closure assembly as claimed in any one of claims 1 to 6 in which the two portions are both of the same plastics material.

9. A closure assembly as claimed in any one of claims 1 to 8 in which the blocking portion is capable of being broken away from the rim portion without deformation of either of the two portions.

10. A closure assembly as claimed in any one of claims 1 to 8 in which the two portions are interlocked with each other so that some deformation of at least one of the parts occurs when breaking away the blocking portion.

11. A closure assembly as claimed in any one of claims 1 to 8 in which, at the moulding interface, an annular surface of one of said portions is conformed with an annular surface of the other portion in the course of moulding said one portion.

12. A closure assembly as claimed in any one of claims 1 to 11 in which, at the moulding interface, the portions intimately contact each other with at least sufficient fusion bonding therebetween to afford sealing engagement.

13. A closure assembly as claimed in any one of claims 1 to 12 including means for resisting removal of the blocking portion so that the blocking portion can only be separated from the rim portion by application of axial load of predetermined magnitude.

14. A closure assembly as claimed in any one of claims 1 to 13 in which the blocking portion is irreversibly removable from the rim portion so that, once the two portions have been separated from one another, they cannot be restored to the same condition of sealing and separation resistance simply by re-inserting the blocking portion into the rim portion.

15. A container sealed against fluid ingress or egress by a closure assembly according to any one of claims 1 to 14.

16. A fluid handling device having a hollow needle for dispensing fluid and a needle retraction assembly for transferring the needle after use into a housing of the device, the housing being provided with a closure assembly as claimed in any one of claims 1 to 14, the aperture defined by the rim portion constituting a needle entry aperture which, prior to operation of the needle retraction assembly, is closed by the blocking portion, the blocking portion being broken away from the rim portion during needle retraction to allow passage of the needle into the housing via said opening.

17. A device as claimed in claim 16 in which the rim portion is integral with the housing.

18. A device as claimed in claim 17 in which the rim portion and housing both comprise polyethylene and/or polypropylene.

19. A bursting disc-type device constituted by a closure assembly as claimed in any one of claims 1 to 14, rupture of the device occuring on breaking away of the blocking portion from the rim portion.

20. A bursting disc-type device as claimed in claim 19 in which the bursting disc is arranged to rupture at a predetermined load determined at least in part, optionally predominantly or substantially entirely, by the strength of fusion bonding between the blocking portion and the rim portion.

21. A bursting disc-type device as claimed in claim 19 in which the blocking portion and rim portion are coupled together by a detent arrangement at said interface.

22. A bursting disc-type device as claimed in claim 21 in which the resistance to separation afforded by the detent arrangement is supplemented by fusion bonding between the blocking portion and the rim portion.

23. A method of producing a bursting disc-type device comprising producing an annular rim portion and moulding a blocking portion in the presence of the rim portion using a surface of the latter to define a moulding boundary for the blocking portion whereby the blocking portion seals the opening in the rim portion and can be broken away from the rim portion upon the application of a predetermined force to the blocking portion.

24. A method for accessing the interior of a hollow body or vessel provided with a closure assembly or device as claimed in any one of claims 1 to 14 and 18 to 22, comprising applying force to the blocking portion to break the same away from the rim portion.
